(19) United States
(12) Patent Application Publication
(10) Pub. No.: US 2022/0395685 A1
(43) Pub. Date: Dec. 15, 2022

(54) DEVICES, SYSTEMS AND METHODS OF MAPPING NEUROMUSCULAR JUNCTIONS FOR BOTULINUM TOXIN INJECTIONS

(71) Applicant: University of Houston System, Houston, TX (US)

(72) Inventor: Yingchun Zhang, Katy, TX (US)

(21) Appl. No.: 17/838,758

(22) Filed: Jun. 13, 2022

Related U.S. Application Data
(60) Provisional application No. 63/209,626, filed on Jun. 11, 2021.

(51) Int. Cl. A61N 1/36; A61N 1/04; A61B 5/389

(52) U.S. Cl. CPC A61N 1/36; A61N 1/0456; A61N 1/36014; A61B 5/389; A61N 1/36139

(57) ABSTRACT

A system for mapping neuromuscular junctions for botulinum neurotoxin (BoNT) injections includes a stimulation electrode and an electromyography (EMG) sensor array including EMG sensors configured to be arranged about a person's face. Each EMG sensor detects muscle activity of a facial muscle of a facial muscle group. An EMG amplifier includes a plurality of input channels. Each input channel receives data of facial muscle activity in the facial muscle group from the EMG sensor array. A computer is in communication with the EMG amplifier. A processor of the computer identifies neuromuscular junctions (NMJs) of the facial muscle group based on the data of facial muscle activity received from the EMG sensor array. The plurality of NMJs are mapped with respect to the at least one facial muscle group of the body of the person. At least one NMJ site for BoNT injection is recommended by the computer.

### CROSS REFERENCE TO RELATED APPLICATION

This U.S. Non-Provisional Patent application claims priority to U.S. Provisional Patent Application No. 63/209,626, filed on Jun. 11, 2021, the entire contents of which are incorporated by reference herein.

### FIELD

The present disclosure relates to botulinum toxin injections, more specifically, to devices, systems, and methods of mapping neuromuscular junctions for botulinum toxin injections.

### BACKGROUND

Botulinum toxin (BoNT) injection for treatment of facial wrinkles is the most frequently performed cosmetic procedure in the United States, and it is one of the most common entry procedures for clinicians seeking to incorporate aesthetic treatments into their practice. Wrinkles are formed by dermal atrophy and repetitive contraction of underlying facial musculature. BoNT is a potent neurotoxin that inhibits release of acetylcholine at the neuromuscular junction (NMJ). Injection of appropriate quantities of NMJs into specific overactive muscles causes localized muscle relaxation that smooths the overlying skin and reduces wrinkles. BoNT effects take about two weeks to fully develop and last three to four months. Treatment of frown lines and crow's feet, which are the cosmetic indications approved by the U.S. Food and Drug Administration (FDA). It has become a common treatment for wrinkles occurring in the upper one-third of the face (i.e., frown lines, horizontal forehead lines, and crow's feet).

BoNT injection has been applied to facial muscles for noncosmetic uses including treatment of spastic facial dystonias, temporary treatment of idiopathic or thyroid dysfunction-induced upper eyelid retraction, suppression of undesired hyperlacrimation, induction of temporary ptosis by chemodenervation in facial paralysis, and correction of lower eyelid spastic entropion. Additional periocular uses include control of synchronic eyelid and extraocular muscle movements after aberrant regeneration of cranial nerve palsies.

### SUMMARY

Provided in accordance with aspects of the present disclosure is a system for mapping neuromuscular junctions for botulinum neurotoxin (BoNT) injections including a stimulation electrode configured to stimulate at least one nerve innervated with a facial muscle group in a body of a person. A stimulation electrode controller is in electrical communication with the stimulation electrode. An electromyography (EMG) sensor array includes a plurality of EMG sensors configured to be arranged about the person's face. Each EMG sensor detects muscle activity of at least one muscle of the facial muscle group. An EMG amplifier is in electrical communication with the stimulation electrode, the stimulation electrode controller and the EMG sensor array. The EMG amplifier includes a plurality of input channels. Each input channel receives data of facial muscle activity in the facial muscle group of the body of the person from the EMG sensor array. A computer is in communication with the EMG amplifier. The computer includes a processor and a memory. The processor identifies a plurality of neuromuscular junctions (NMJs) of the facial muscle group of the body of the person based on the data of facial muscle activity received from the EMG sensor array. The plurality of NMJs are mapped with respect to the at least one facial muscle group of the body of the person. At least one NMJ site for BoNT injection is recommended by the computer.

In an aspect of the present disclosure, the EMG sensors of the EMG sensor array are arranged on a flexible substrate configured to conform to contours of the person's face. The flexible substrate is formed of silicone.

In an aspect of the present disclosure, each EMG sensor array is a flexible surface EMG sensor array.

In an aspect of the present disclosure, the data of facial muscle activity includes data of at least one overactive facial muscle. The data of facial muscle activity includes a quantitative assessment of severity of overactivity in the at least one overactive facial muscle.

In an aspect of the present disclosure, the recommendation of at least one NMJ site for BoNT injection includes a recommendation of a particular BoNT dose.

In an aspect of the present disclosure, the recommendation of at least one NMJ site for BoNT injection includes a recommendation of a dosing schedule for BoNT.

In an aspect of the present disclosure, the EMG sensor array is configured to detect passive muscle activity and identify an occurrence of at least one spontaneous facial muscle spasm of a facial muscle group of the body of the person.

In an aspect of the present disclosure, the computer is wirelessly or cable connected with the EMG amplifier.

In an aspect of the present disclosure, the computer is included in a smartphone or tablet computer.

Provided in accordance with aspects of the present disclosure is a method for mapping neuromuscular junctions for BoNT injections including stimulating at least one nerve innervated with at least one facial muscle group in a body of a person. The method includes detecting muscle activity of at least one muscle of the at least one facial muscle group. The method includes receiving facial muscle activity in the at least one facial muscle group. The method includes mapping NMJs of the at least one facial muscle group of the body of the person based on the data of facial muscle activity received from the EMG sensor array. The method includes recommending at least one NMJ site for BoNT injection.

In an aspect of the present disclosure, the method includes recommending BoNT dosage for each BoNT injection.

### BRIEF DESCRIPTION OF THE DRAWINGS

Various aspects and features of the present disclosure are described hereinbelow with reference to the drawings wherein:

FIG. 1 is a schematic illustration of a system for neuromuscular junction mapping according to aspects of the present disclosure;

FIG. 2 illustrates various flexible surface EMG array configurations usable by the system of FIG. 1 according to aspects of the present disclosure;

FIG. 3A illustrates exemplary placements of flexible surface EMG arrays for use of the system of FIG. 1;

FIG. 3B illustrates an exemplary placement of a stimulation electrode for use of the system of FIG. 1;

FIG. 4 is a block diagram of a method for neuromuscular junction mapping according to aspects of the present disclosure; and

FIG. 5 is a block diagram of an exemplary computer of the system of FIG. 1 according to aspects of the present disclosure.

### DETAILED DESCRIPTION

Descriptions of technical features or aspects of an exemplary configuration of the disclosure should typically be considered as available and applicable to other similar features or aspects in another exemplary configuration of the disclosure. Accordingly, technical features described herein according to one exemplary configuration of the disclosure may be applicable to other exemplary configurations of the disclosure, and thus duplicative descriptions may be omitted herein.

Exemplary configurations of the disclosure will be described more fully below (e.g., with reference to the accompanying drawings). Like reference numerals may refer to like elements throughout the specification and drawings.

The effectiveness of botulinum neurotoxin (BoNT) injections is a at least partially dependent on delivery of an appropriate dose to an appropriate injection site. Accurate localization of a BoNT injection to a desired neuromuscular junction (NMJ), for example, can minimize an effective dose and reduce or eliminate any known clinical side effects of BoNT injections. BoNT treatment has had problems with variable clinical effectiveness and high treatment cost. As BoNT acts at NMJs, injection directed to the proximity of NMJs will retain and potentiate the treatment efficacy while reducing dosage and consequently minimizing adverse effects and treatment cost. Injecting BoNT to non-NMJ locations requires larger injecting dosage compared to NMJ-target injection, as the injection needs to defuse to NMJs to be effective. Studies have demonstrated that increasing the injection distance by as little as 1 cm from the NMJ reduced the effect of BoNT by 46%.

Efforts have been taken to localize NMJ locations, however, there is no technology currently available for accurately localizing NMJ by eliminating the blurring effect caused by low-conductive fat and skin tissues, and the interfering effects (cross talk) caused by neighboring muscles. Furthermore, there is no technology currently available for correlating the severity assessment for specific overactive muscles with their NMJ locations to guide personalized precision BoNT injection.

The minimal amplitude channel and/or the channel with phase reversal in the single differential signal may be utilized to identify the surface NMJ locations. The NMJs can be localized over the skin surface through visual inspections or automatic algorithms such as bi-dimensional cross correlation, template matching or optical flow. Those surface innervation zone (IZ) mapping techniques can also be used to localize IZs over the skin surface to improve the BoNT injection outcome, but the application of those surface localization methods can be limited by the blurring effects caused by low-conductive fat and skin tissues, and the interfering effect (cross talk) caused by neighboring muscles. Clinically, the motor point (MP) is used as the injection site of BoNT since it is homologous to the IZ. However, a significant difference between the MP and IZ locations has been observed. There is an unmet need in both technical (the need for a precision and clinically friendly NMJ mapping and overactive muscle severity assessment technique) and clinical terms (the need for NMJ distribution patterns in facial muscles for precision BoNT injection with personalized injection sites and optimized injection dosage).

Aspects of the present disclosure provide a novel transcutaneous NMJ mapping technique to accurately localize NMJ locations from multi-channel surface EMG signals recorded during spontaneous activity or under nerve stimulation.

Aspects of the present disclosure provide a novel muscle overactivity severity assessment technique to quantitatively assess the severity of overactive muscles. By employing guided precision BoNT injections to facial muscles for both cosmetic and non-cosmetic indications, and by personalizing BoNT injection sites using NMJ mapping results and personalizing BoNT injection dosage using muscle overactivity severity assessment results, improved BoNT injection treatment outcomes with reduced treatment cost and minimized side effects are achieved.

The devices, systems and methods described herein fulfill the current clinical and research needs for precision injection of BoNT into the proximity of NMJs of overactive facial muscles for the best treatment outcome with minimized dosage and side effects. The devices, systems and methods described herein employ a flexible surface EMG array in a variety of configurations with different number of electrode number and shapes to meet the needs of recording muscle activity for different facial muscle groups, and an electrical simulator which will be used to deliver electrical stimulations to at least one facial nerve.

The flexible surface array detects complete and detailed neuromuscular information of any specific facial muscle groups of interest with the appropriate choice of the configuration. The flexible surface arrays may be customized for each specific facial muscle of interest, and may be customized to meet individual facial contours. Surface EMG signals can be collected at rest and/or during voluntary contraction for muscle overactivity severity assessment, and during facial nerve stimulation for NMJ mapping. Surface EMG signals may be amplified and digitized by an amplifier and analog to digital (A/D) converter for online and offline single processing via a processing computer. Multi-channel surface EMG may be used for multiple neuromuscular function evaluation techniques with monopolar or differential setups such as myoelectric source imaging, NMJ identification, surface EMG decomposition and motor unit number estimation. For example, minimal or no response to facial nerve stimulation reveal innervation deficit; large variability of firing rate of motor units during voluntary may suggest compromised central regulation of muscle functions; loss of motor unit number may indicate neural degeneration or axonal injury.

The devices, systems and methods described herein can be employed to guide precision BoNT injections to facial muscles for both cosmetic and non-cosmetic indications, by personalizing BoNT injection sites using NMJ mapping results and personalizing BoNT injection dosage using muscle overactivity severity assessment results, to maximize and stabilize the BoNT injection treatment outcome with reduced treatment cost and minimized side effects.

The devices, systems and methods described herein can be employed to personalize BoNT injection for both cosmetic purpose (reducing the appearance of facial wrinkles) and non-cosmetic purpose (treating Blepharospasm, Strabismus, spastic facial dystonias and other facial muscle spasms).

The devices, systems and methods described herein can be employed to quantitatively assess nerve innervation for pre-surgery planning, pre/post-surgery evaluation, surgery monitoring, and other neuromuscular function and innervation assessment associated with disease diagnosis, disease progress and treatment outcome evaluation. The devices, systems and methods described herein can also be employed for developing treatment regimens of BoNT injections and monitoring the progress of such treatment regimens in terms of desired clinical outcomes.

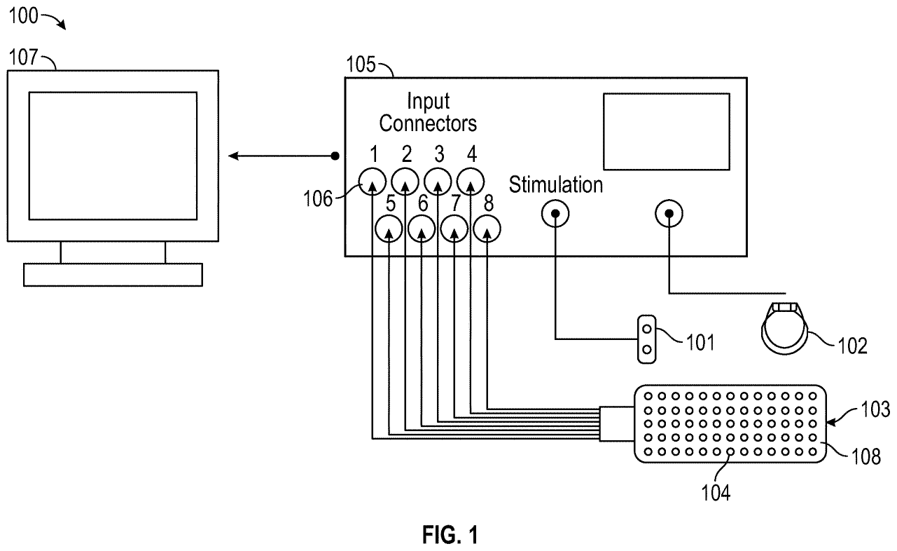

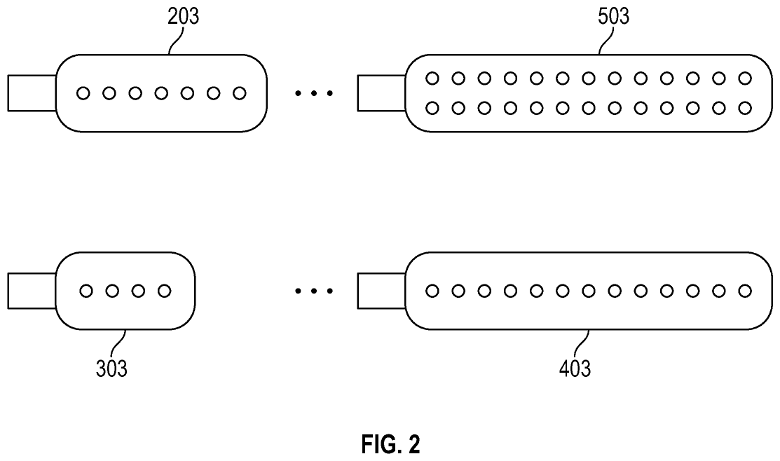

FIGS. 1 and 2 illustrate a nerve stimulation and flexible multi-channel surface EMG array evaluation system 100 according to aspects of the present disclosure. The system 100 can be employed for precision BoNT injections to facial muscles for both cosmetic and non-cosmetic indications. FIG. 2 illustrates various flexible surface EMG array configurations (203, 303, 403 and 502) usable by the system of FIG. 1

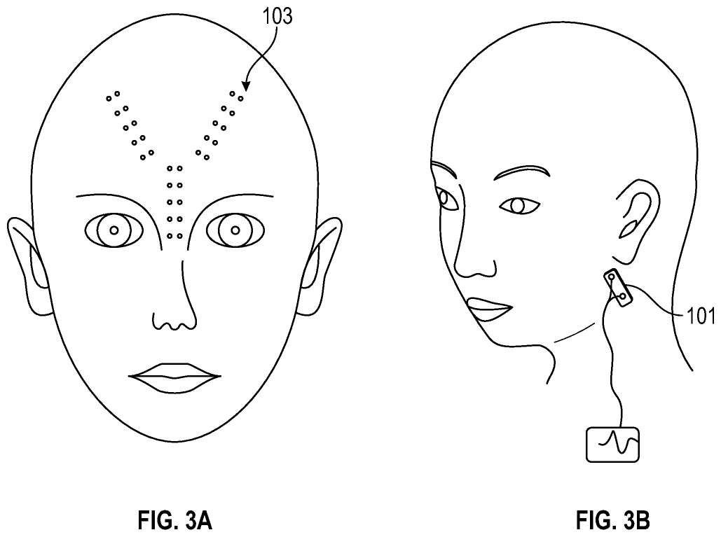

FIGS. 3A and 3B illustrates exemplary positioning and use of the system in mapping the distribution of neuromuscular junctions of facial muscles and assessing the severity of overactive facial muscles, using the procerus and frontalis muscles as examples.

Referring to FIGS. 1-3, a system 100 for mapping neuromuscular junctions for botulinum neurotoxin (BoNT) injections including a stimulation electrode 101 configured to stimulate at least one nerve innervated with a facial muscle group in a body of a person. A stimulation electrode controller 102 is in electrical communication with the stimulation electrode 101. As an example, the stimulation electrode controller 102 may be a pedal controller, such as a pedal controller manually operated by a physician.

According to an aspect of the present disclosure, the stimulation electrode 102 may execute a pre-programmed stimulation program including a series of intermittent electrical pulses controlled by the computer 107 described herein and delivered to at least one nerve innervated with at least one facial muscle group.

An electromyography (EMG) sensor array 103 includes a plurality of EMG sensors 104 configured to be arranged about the person's face. Each EMG sensor 104 detects muscle activity of at least one muscle of the facial muscle group. For example, each EMG sensor 104 is a subcutaneous EMG sensor for measuring muscle activity through a person's skin when the EMG sensor array is arranged about a person's face.

The EMG sensors 104 of the EMG sensor array 103 are arranged on a flexible substrate 108 configured to conform to contours of the person's face. The flexible substrate 108 may be formed of or may include silicone. The flexible substrate 108 may include an adhesive side that faces a person's face and is used to secure the flexible substrate to the person's face.

The EMG sensors 104 of the EMG sensor array 103 can detect an initial action potential elicited in a particular region of a facial muscle and can detect a propagation of actional potentials across the facial muscle in a particular direction. Data of the direction of action potential propagation can be utilized to identify a particular muscle by cross-referencing the direction of action potential propagation with a known arrangement of facial muscles. As an example, the computer 107 may store a reference database of known facial muscles including data of a size and directional orientation of such facial muscles employable by the computer for cross-referencing to identify a facial muscle. A machine learning algorithm may be employed to identify a particular facial muscle based on a directional propagation profile of detected actional potentials.

An EMG amplifier 105 is in electrical communication with the stimulation electrode 101, the stimulation electrode controller 102 and the EMG sensor array 103. The EMG amplifier 105 includes a plurality of input channels 106. Each input channel 106 receives data of facial muscle activity in the facial muscle group of the body of the person from an EMG sensor 104 of the EMG sensor array 103.

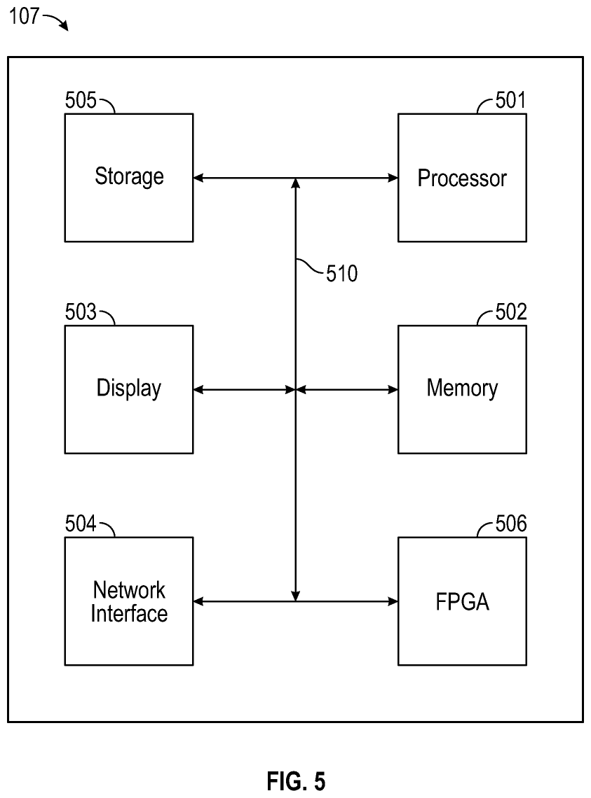

A computer 107 (see, e.g., FIGS. 1 and 5) is in communication with the EMG amplifier 105. The computer 107 receives EMG sensor data from each EMG sensors 104 of the EMG sensor array 103. The computer 107 includes a processor 501 and a memory 502. The memory 502 stores computer instructions (e.g., software) configured to be executed by the processor 501 to perform a series of algorithms for identifying a plurality of neuromuscular junctions (NMJs) of the facial muscle group of the body of the person based on the data of facial muscle activity received from the EMG sensor array 103. The plurality of NMJs are mapped by the computer 107 with respect to the at least one facial muscle group of the body of the person. The mapped NMJs may be displayed on a display device 503 of the computer 107. At least one NMJ site for BoNT injection is identified and recommended by the computer 107.

The data of facial muscle activity includes data of at least one overactive facial muscle. The data of facial muscle activity includes a quantitative assessment of severity of overactivity in the at least one overactive facial muscle. Thus, a corresponding NMJ site of the overactive facial muscle that is identified may be recommended as a desired NMJ injection site for BoNT. The recommendation of at least one NMJ site for BoNT injection may include a recommendation of a particular BoNT dose. Additionally, the recommendation of at least one NMJ site for BoNT injection may include a recommendation of a dosing schedule for BoNT.

In an aspect of the present disclosure, the EMG sensor array 103 is configured to detect passive muscle activity and identify an occurrence of at least one spontaneous facial muscle spasm of a facial muscle group of the body of the person. The EMG sensor array 103 may also detect voluntary facial muscle movements.

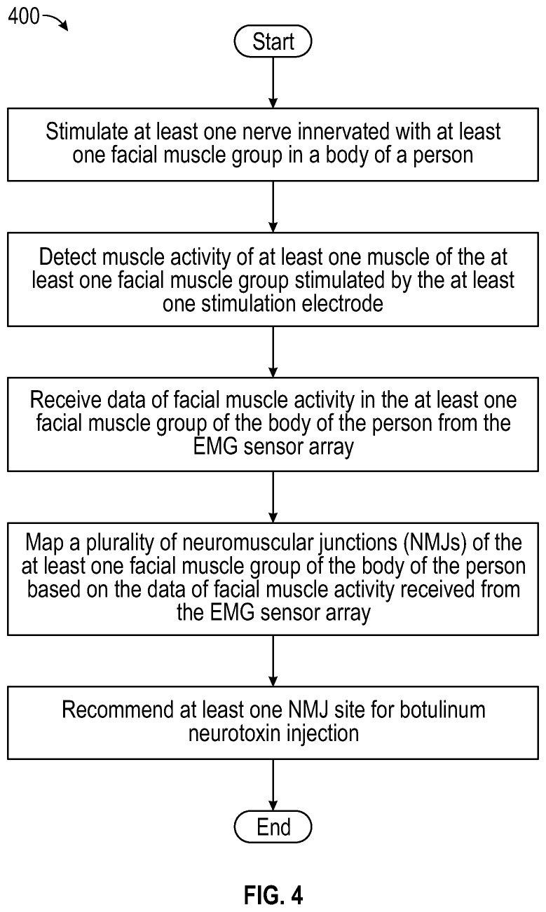

Referring to FIG. 4, a method 400 for mapping neuromuscular junctions for BoNT injections includes stimulating, by the stimulation electrode, at least one nerve innervated with at least one facial muscle group in a body of a person. The method 400 includes detecting, by the EMG sensor array including the EMG sensors arranged about the person's face, muscle activity of at least one muscle of the at least one facial muscle group stimulated by the at least one stimulation electrode. The method 400 includes receiving, at the EMG amplifier, data of facial muscle activity in the at least one facial muscle group of the body of the person from the EMG sensor array. The method 400 includes mapping, by a computer including a processor and a memory (see, e.g., FIG. 5), a plurality of NMJs of the at least one facial muscle group of the body of the person based on the data of facial muscle activity received from the EMG sensor array. The method 400 includes recommending, by the computer, at least one NMJ site for BoNT injection.

In an aspect of the present disclosure, the method 400 described with reference to FIG. 4 may include assessing overactivity and/or spasms occurring in the facial muscle group of the body of the person based on the data of facial muscle activity received from the EMG sensor array. Data may be recorded, for example, at rest, during voluntary contraction and/or under nerve stimulation, as described herein.

FIG. 5 is a block diagram of an exemplary computer 107 of the system of FIG. 1 according to aspects of the present disclosure. The computer 107 described with reference to FIG. 5 may perform the steps described with reference to FIG. 4.

Referring to FIG. 5, the computer 107 may include a processor 501 connected to a computer-readable storage medium or a memory 502 which may be a volatile type memory, e.g., RAM, or a non-volatile type memory, e.g., flash media, disk media, etc. The processor 501 may be another type of processor such as, without limitation, a digital signal processor, a microprocessor, an ASIC, a graphics processing unit (GPU), field-programmable gate array (FPGA), or a central processing unit (CPU).

In some aspects of the disclosure, the memory 502 can be random access memory, read-only memory, magnetic disk memory, solid state memory, optical disc memory, and/or another type of memory. The memory 502 can communicate with the processor 501 through communication buses 510 of a circuit board and/or through communication cables such as serial ATA cables or other types of cables. The memory 502 includes computer-readable instructions that are executable by the processor 501 to operate a control unit embodying the computer 107. The computer may 107 include a network interface 504 (e.g., a wireless network interface) to communicate with other computers or a server. A storage device 505 may be used for storing data. The computer 107 may include one or more FPGAs 506. The FPGA 506 may be used for executing various machine learning algorithms.

In an aspect of the present disclosure, the computer 107 is wirelessly or cable connected with the EMG amplifier 105. Alternatively, the computer 107 may be connected with the EMG amplifier 105 via a wired connection (e.g., a USB connection). As an example, the computer 107 may be included in a smartphone or tablet computer. The computer may also be a laptop or desktop computer in communication with the EMG amplifier 105.

It will be understood that various modifications may be made to the aspects and features disclosed herein. Therefore, the above description should not be construed as limiting, but merely as exemplifications of various aspects and features. Those skilled in the art will envision other modifications within the scope and spirit of the claims appended thereto.

What is claimed is:

1. A system for mapping neuromuscular junctions for botulinum neurotoxin (BoNT) injections, comprising:
at least one stimulation electrode configured to stimulate at least one nerve innervated with at least one facial muscle group in a body of a person;
at least one stimulation electrode controller in electrical communication with the at least one stimulation electrode, the at least one stimulation electrode controlled configured to control the at least one stimulation electrode;
an electromyography (EMG) sensor array including a plurality of EMG sensors configured to be arranged about the person's face, each EMG sensor of the plurality of EMG sensors configured to detect muscle activity of at least one muscle of the at least one facial muscle group stimulated by the at least one stimulation electrode;
an EMG amplifier in electrical communication with the at least one stimulation electrode, the at least one stimulation electrode controller and the EMG sensor array, the EMG amplifier including a plurality of input channels, each input channel of the plurality of input channels configured to receive data of facial muscle activity in the at least one facial muscle group of the body of the person from the EMG sensor array; and
a computer in communication with the EMG amplifier, the computer including a processor and a memory, the memory storing computer instructions configured to be executed by the processor, the computer instructions configured to instruct the processor to identify a plurality of neuromuscular junctions (NMJs) of the at least one facial muscle group of the body of the person based on the data of facial muscle activity received from the EMG sensor array, map the plurality of NMJs with respect to the at least one facial muscle group of the body of the person, and recommend at least one NMJ site for BoNT injection.

2. The system of claim 1, wherein the plurality of EMG sensors of the EMG sensor array are arranged on a flexible substrate configured to conform to contours of the person's face.

3. The system of claim 1, wherein the flexible substrate is formed of silicone.

4. The system of claim 1, wherein each EMG sensor of the plurality of EMG sensors is a transcutaneous EMG sensor.

5. The system of claim 1, wherein the data of facial muscle activity includes data of at least one overactive facial muscle.

6. The system of claim 5, wherein the data of facial muscle activity includes a quantitative assessment of severity of overactivity in the at least one overactive facial muscle.

7. The system of claim 1, wherein the recommendation of at least one NMJ site for BoNT injection includes a recommendation of a particular BoNT dose.

8. The system of claim 1, wherein the recommendation of at least one NMJ site for BoNT injection includes a recommendation of a dosing schedule for BoNT.

9. The system of claim 1, wherein the EMG sensor array is configured to detect passive muscle activity and identify an occurrence of at least one spontaneous facial muscle spasm of the at least one facial muscle group of the body of the person.

10. The system of claim 1, wherein the computer is wirelessly or cable connected with the EMG amplifier.

11. The system of claim 1, wherein the computer is included in a smartphone or tablet computer.

12. A method for mapping neuromuscular junctions for botulinum neurotoxin (BoNT) injections, comprising:
stimulating, by at least one stimulation electrode, at least one nerve innervated with at least one facial muscle group in a body of a person;
detecting, by an electromyography (EMG) sensor array including a plurality of EMG sensors arranged about the person's face, muscle activity of at least one muscle of the at least one facial muscle group stimulated by the at least one stimulation electrode;
receiving, at an EMG amplifier, data of facial muscle activity in the at least one facial muscle group of the body of the person from the EMG sensor array;
mapping, by a computer including a processor and a memory, a plurality of neuromuscular junctions (NMJs) of the at least one facial muscle group of the body of the person based on the data of facial muscle activity received from the EMG sensor array; and
recommending, by the computer, at least one NMJ site for BoNT injection.

13. The method of claim 12, wherein the EMG sensor array if a flexible EMG sensor array, and wherein the method includes arranging the flexible EMG sensor array to conform to contours of the person's face.

14. The method of claim 12, wherein the EMG sensors of the EMG sensor array detect muscle activity transcutaneously.

15. The method of claim 12, wherein the data of facial muscle activity received at the EMG amplifier includes data of at least one overactive facial muscle.

16. The method of claim 15, further including performing, by the computer, a quantitative assessment of severity of overactivity in the at least one overactive facial muscle.

17. The method of claim 12, further including recommending, by the computer, a particular BoNT dose.

18. The method of claim 12, further including recommending, by the computer, a dosing schedule for BoNT.

19. The method of claim 12, further including detecting, by the EMG sensor array, passive muscle activity and identifying an occurrence of at least one spontaneous facial muscle spasm of the at least one facial muscle group of the body of the person.

20. The method of claim 12, further including wirelessly or cable transmitting the data of facial muscle activity in the at least one facial muscle group of the body of the person from the EMG sensor array to the computer.
